(19) United States
(12) Patent Application Publication
(10) Pub. No.: US 2022/0151758 A1
(43) Pub. Date: May 19, 2022

(54) BIO-FUNCTIONALIZED PROSTHETIC STRUCTURE WITH CORE-SHELL ARCHITECTURE FOR PARTIAL OR TOTAL REPAIR OF HUMAN TENDONS OR LIGAMENTS

(71) Applicants: UNIVERSIDADE DO PORTO, Porto (PT); UNIVERSIDADE DO MINHO, Braga (PT)

(72) Inventors: Maria Ascensao FERREIRA DA SILVA LOPES, Porto (PT); Diana Raquel DOS SANTOS MORAIS, Porto (PT); Rui Jorge SOUSA COSTA DE MIRANDA GUEDES, Porto (PT); Raul Manuel ESTEVES DE SOUSA FANGUEIRO, Guimaraes (PT); Juliana Patricia CORREIA DA CRUZ, Guimaraes (PT); Tiago DE MELO SILVA RAMOS PEREIRA, Porto (PT); Joao Manuel DA COSTA FERREIRA TORRES, Porto (PT)

(21) Appl. No.: 17/598,616

(22) PCT Filed: Mar. 30, 2020
(86) PCT No.: PCT/IB2020/053014

§ 371 Date: Sep. 27, 2021

(30) Foreign Application Priority Data
Mar. 28, 2019 (PT) 115407

(51) Int. Cl. A61F 2/00; A61F 2/08; A61L 27/18; A61L 27/34; A61L 27/56; A61L 27/58

(52) U.S. Cl. CPC A61F 2/0077; A61F 2/08; A61L 27/18; A61L 27/34; A61L 27/56; A61L 2430/10; A61F 2002/009; A61F 2250/0051; A61F 2250/0056; A61L 2400/18; A61L 27/58

(57) ABSTRACT

The present invention relates to a bio-functionalized fibrous structure with a core/shell architecture for partial or total repair of human tendons or ligaments. The architecture based on a core/shell system grants to the fibrous structure a specific physical and mechanical behaviour when it is repeatedly mechanically loaded, as happens with a native tendon or ligament in constant usage in the human body. The core is based on several sub-components, namely braided structures parallelly assembled, which are enclosed by a braided shell. Additionally, a selective bio-functionalization of the two parts of the core/shell structure can be applied in order to selectively improve or avoid the in vivo cell adhesion.

### TECHNICAL DOMAIN

The present invention relates to a bio-functionalized fibrous structure with a core/shell architecture for partial or total repair of human tendons or ligaments.

### BACKGROUND OF THE INVENTION

Tendons/ligaments present a complex mechanical behaviour due to the complex hierarchical collagen fibrous structures, having as primary function the transmission of tensile forces from a muscle to a bone or bone to bone respectively, and acting as a buffer by absorbing external excessive forces to prevent muscle damage. Tendons/ligaments response to load is non-linear and anisotropic, presenting high mechanical strength, good flexibility and a viscoelastic behaviour, due to the viscous properties of the collagen fibres and ground substance, exhibiting force-relaxation, creep and mechanical hysteresis.

A typical stress-strain curve of an isolated tendon/ligament, in elongation-to-failure conditions, presents three different regions. In the first region, named as toe region, small forces result in a large lengthening due to the crimped collagen fibrous nature, and when the stress is released the crimped pattern and tendon length are restored. The toe region typically ends at about 1.5%-3.0% strain. In case of further elongation, a second region, named as linear region, appears with constant and higher stiffness (curve slope). In general tendons and ligaments can be strained to between 5 and 7% without damage. However, in ligaments with very high elastin content can be strained up to 30% or more without damage. After this region, if the elongation continues, collagen fibres start to fail in an unpredictable way causing tears in the tissue, leading to the total rupture. The maximum strain before failure is generally in the neighbourhood of 12-15%.

The maximum force, maximum strain, stiffness and Young's modulus depend on the thickness and collagen content of the tendon or ligament type, patient gender, age and physical activity.

In general, the ultimate tensile strengths for tendons and ligaments range from 50 to 150 MPa and the elastic modulus values reported range between 1 and 2 GPa.

The healing of tendons/ligaments after an injury, is a very slow and inefficient process which never restores the biological and biomechanical properties completely. This process requires to re-establish the tendon/ligament fibres and structure, and the gliding mechanism between the tendon/ligament and the surrounding structures.

Currently, tendon/ligament injuries, whether acute or chronic, are usually managed using two approaches, conservative, surgical or simultaneously both. The conservative management, used as the first approach in some clinical cases of low degree injury to relief the pain, involves rest, mechanical conditioning, corticosteroids injection, orthotics, ultrasound, laser or shockwave treatment. However, due to the limited tendon ability for self-healing in same injury cases, this recovery approach requires long treatment periods, potential partial function loss and recurrent injury, failing in many cases. So, when this approach does not result or is not appropriate attending to the extension of the lesion, such as in cases of total rupture, surgical intervention is used, suturing the injured ends together or fixing the tendon to the bone. But, in many cases this approach can also tail due to the poor healing ability of the degenerated tissue involved, which even after healing presents a loss of mechanical performance compared to the native tissue, being susceptible to rupture again and a repeated surgery is required.

Cell growth and function are influenced by the biomaterial surface characteristics, such as morphology and physical and chemical features. For instance, the materials' surface roughness and wettability can influence the type and the adsorption kinetics of the serum proteins to the material surface. The adsorbed protein layer has an essential role in the cell adhesion, morphology and migration, because the charged cell membrane interacts with surfaces through this protein layer. It has been reported in several studies that a certain level of roughness and hydrophilicity, as well as the functionalization of surfaces with specific functional groups, such as (—OH) and (—NH₂), favour the adsorption of that protein layer and consequently the cell adhesion.

The chemical grafting of specific functional groups on polymeric scaffolds surface, namely of amino groups (NH2), has been studied based on a chemical etching in the form of aminolysis by a condensation reaction using diamines, such as ethylenediamine (EDA). Aminolysis has been presented as effective to modify polymeric scaffolds for tissue engineering applications, where the free amino groups were used as a chemical linker to immobilize macromolecules, such as gelatin, chitosan and collagen, or they can directly interact with the extracellular matrix (ECM). The (—NH₂) groups from EDA can be chemisorbed on polymeric substrates via (—C(O)NH) bonds, which result from the reaction between an ester group available in the polymer and one amine side only from EDA, leading to amide formation. The other amine side is available to interact with the ECM molecules, improving the interface between the scaffolds surface and surrounding cells. The several amino groups presented on the material surface present positive charges which are able to establish electrostatic interactions with the negative charges of cell-surface proteins, promoting the adhesion of cells to the material.

In some clinical cases, attending to the extensive damage, in spite of the several drawbacks associated to biological grafts they may be required to replace the damaged tendon. Autografts are only available in limited amounts, can induce morbidity, tissue laxity, poor tissue integration and functional disability at the donor site. Allografts and xenografts are not recommended once they may cause a harmful response from the immune system, causing rejection, and present the risk of disease transmission.

Therefore, due to the limitations of these treatment approaches, finding suitable scaffolds to promote the tissue regeneration in vivo, or even an artificial tendinous tissue by association of cells and growth factors to those devices in vitro, using a tissue engineering approach, is nowadays a clinical challenge. In the last years, some commercial scaffolds, in the form of patches, have been used to provide some protection in case of soft tissue tears or to provide some mechanical support when associated with grafts. Besides that, to accomplish a treatment solution for extensive or total damage of the tissue, other scaffolds are being developed by researchers to be used as prosthetic devices to partially or fully replace a tendon.

Document US2017273775 A1 discloses a three-dimensional braided scaffold produced directly from filaments while in the case of the present application the core-shell structure is produced from braids made by filaments. That is, in the case of this document each of the filaments that form the final braided structure were not previously braided and then the final structure produced as is the case of the present technology. Therefore, the mechanical behaviour of both structures is completely different being the behaviour of the present core-shell structure is controlled at two levels: of the various braids that give rise to the core-shell and the structure itself. The blocking point of the fibrous structure, which corresponds to the point of significant increase in stiffness, is therefore controlled at these two levels. The woven structure presented in this document presents a fibrous architecture completely different from that recommended in the present application. Thus, while in the present application the core and the shell configure two layers with no connection between them, the structure described in the document presents filament/threads that orient themselves from the outer layer to the inner layer, crossing the entire structure, connecting it. In this case, the mechanical behaviour is significantly different from that described herein in which the external structure (shell) is responsible, in the first stage, for the low rigidity of the shell, and the internal structure (core) when requested after partial deformation of the external structure is responsible for the high stiffness presented after the blocking point of the structure.

This document does not conflict with the present technology because it uses filaments with a different structure to produce the shell and the shell structure itself is also different, presenting different mechanical behaviour.

Document “Hybrid core-shell scaffolds for bone tissue engineering”, Biomedical Materials, vol. 14, Number 2 (2019), discloses a structure developed for the application in bone regeneration while the present structure is for regeneration of tendons and ligaments. In this document, the core and shell structures are tubular structures with a hollow core whereas the core of the present technology is composed of braids.

The shell contains hydroxyapatite to promote bioactivity and attract cells. The present technology was designed to have the opposite behaviour, i.e. a shell with anti-adherent properties to avoid adherences which are a major clinical problem in tendon/ligament regeneration.

The fibrous core-shell structure is produced by a coaxial electrospinning so the obtained fibrous structure is completely different from the present application, since the one produced in this document presents nanofibers with random orientation in a fibrous mantle, and the one proposed herein presents braided filaments that are later transformed into a rope with orientation at well-defined angles.

Document US2007255422 A1 discloses a structure developed for the application in bone regeneration while the present structure is for regeneration of tendons and ligaments. The structure in the document includes a core and a sheath which are bonded by a compression moulding process leading to obtaining a rigid structure and therefore the final fibrous structure is completely different from the one herein described. The fact that the polymeric yarns are bonded limits their deformation capacity, which compromise the need for satisfaction of the three phases of tensile behaviour typical of natural tendons and ligaments. The solution recommended in the present application presents a fibrous structure that can move freely during their deformation to the point of blocking the structure itself. This behaviour will not be achieved from the rigid structures protected by the present patent.

### SUMMARY

The present application relates to a bio-functionalized prosthetic structure with core-shell architecture for partial or total repair of human tendons or ligaments with:
the core comprises braided structures parallelly assembled based on a plurality of biocompatible polymeric filaments;
   the core structure comprises a braid angle from 0 to 90°;
   the core has a diameter of up to 2 cm;
the shell encloses the core and is a braided structure based on a plurality of biocompatible polymeric filaments;
   the shell structure comprising a braid angle from 0 to 90°;
   the shell has a thickness of up to 5 mm;

wherein the biocompatible polymeric filaments and/or braids in the core comprise a bioactive surface treatment suitable for cell adhesion and proliferation;

wherein the biocompatible polymeric filaments and/or braids in the shell comprise a biopassive surface treatment suitable to avoid the formation of adhesion plates between the prosthetic structure and the surrounding tissues of tendons or ligaments.

In one embodiment the biocompatible polymeric filaments in the core are composed by non-degradable filaments, such as of polypropylene (PP), polyethylene (PE), poly (ethylene terephthalate) (PET), polyamide (PA), any reinforced composite based on any of these polymers or by any combination thereof.

In another embodiment the biocompatible polymeric filaments in the core are composed by biodegradable filaments, such as polydioxanone (PDO), poly(glycolic-co-caprolactone) (PGCL), poly(glycolic-co-lactic acid) (PGLA), poly(lactic acid) (PLA), poly(lactic-co-glycolic acid) (PLGA), any reinforced composite based on any of these polymers or by any combination thereof.

In yet another embodiment the biocompatible polymeric filaments in the shell are composed by non-degradable filaments, such as polypropylene (PP), polyethylene (PE), poly (ethylene terephthalate) (PET) or even polyamide (PA), any reinforced composite based on any of these polymers or by any combination thereof.

In another embodiment the biocompatible polymeric filaments in the shell are composed by biodegradable filaments selected from the group of polydioxanone (PDO), poly(glycolic-co-caprolactone) (PGCL), poly(glycolic-co-lactic acid) (PGLA), poly(lactic acid) (PLA), poly(lactic-co-glycolic acid) (PLGA), 5Poly(3-hydroxybutyrate-co-3 hydroxyhexanoate) (PHBHHx), poly(3-hydroxybutyrate) (PHB), Polycaprolactone (PCL), Poly(lactic acid; (PLAs), any reinforced composite based on any of these polymers or by any combination thereof.

In one embodiment the diameter of the filaments is within the range of 5-1000 μm.

In another embodiment the bioactive surface treatment is based on grafting —NH₂ groups on the filaments or braids surface of the biocompatible polymeric.

In another embodiment the bioactive surface treatment is based on any functional group grafting after a surface treatment that grants —OH or deprotonated —OH groups to the polymeric structure.

In yet another embodiment the biopassive surface treatment is based on a polytetrafluoroethylene-based coating, or any perfluoro-polymer coating.

In one embodiment the braiding patterns are diamond 1/1 repeat, regular 2/2 repeat or Hercules 3/3 repeat or any derivative.

In another embodiment the biopassive surface treatment is based on a superhydrophobic (contact angle ≥150°) or a superhydrophilic (contact angle ≤5°) compounds.

In one embodiment the braids are biaxial or triaxial.

### BRIEF DESCRIPTIONS OF DRAWINGS

For easier understanding of this application, figures are attached that represent embodiments which nevertheless are not intended to limit the technique disclosed herein.

FIG. 1 shows a cross section of the core/shell prosthetic structure of the present application show-ng the core (1); shell (2), core braids (3), shell braids (4).

FIG. 2 shows a representation of the braids that constitute the core architecture of the present technology.

FIG. 3 shows a representation of the braids that constitute the shell architecture of the present technology.

FIG. 4 shows the experimental data obtained for the bioactive and biopassive treatments in untreated and treated PET braids and yarns.

### DETAILED DESCRIPTION OF THE INVENTION

The present invention provides a functionalized fibrous structure with an architecture based on a core/shell system produced using a fibrous technology-based technique or additive manufacturing. The structure is intended to be used for partial or total repair of any human tendon or ligament.

Currently, tendon and ligament injuries, whether acute or chronic, are usually managed using two approaches: conservative, surgical or simultaneously both. The conservative management, used as the first approach in some clinical cases of low degree injury to relief the pain, involves rest, mechanical conditioning, corticosteroids injection, orthotics, ultrasound, laser or shockwave treatment. However, due to the limited tendon/ligament ability for self-healing in same injury cases, this recovery approach requires long treatment periods, potential partial function loss and recurrent injury, failing in many cases. So, when this approach does not result or is not appropriate attending to the extension of the lesion, such as in cases of total rupture, surgical intervention is used, suturing the injured ends together or fixing the tendon to the bone. But, in many cases this approach can also fail due to the poor healing ability of the degenerated tissue involved, which even after healing presents a loss of mechanical performance compared to the native tissue, being susceptible to rupture again and a repeated surgery is required.

Therefore, due to the limitations of these treatment approaches, finding suitable scaffolds to promote the tissue regeneration in vivo even by association of cells and growth factors to those devices in vitro, using a tissue engineering approach, is nowadays a clinical challenge.

Therefore, the functionalized textile structure discussed in the present disclosure may be used for partial or total substitution of human tendons or ligaments when there is a large extension injury of those tissues and the usually used conservative or surgical approaches are not efficient enough for an appropriate patient recovery. Depending on the injury extension, the developed device may be used just to partially replace the tendon/ligament, being inserted for example between two tendon ends or a tendon end and muscle end, or in more extreme and rare cases it may be needed to fully replace the tendon linking a muscle to a bone.

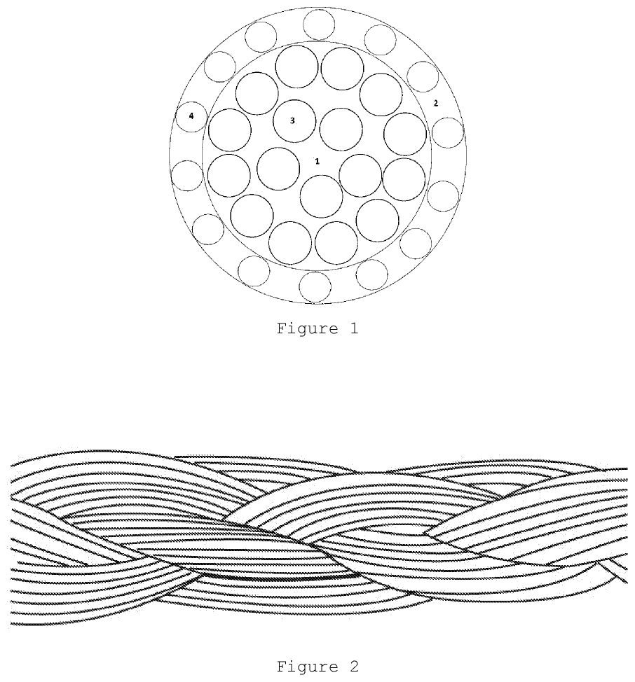

The main advantages of the developed technology are:
The appropriate mechanical performance, stress-strain curve shape, failure load and strain, stiffness, fatigue and creep resistance, to properly replace the physical and mechanical function of a native tendon or ligament for a long-term;
The architecture parameters of the structure may be adapted according to the tendon or ligament intended to be substituted depending on its physical and mechanical features;
The selective bio-functionalization of the two parts of the structure (core (1) and shell (2), FIG. 1) in order to selectively improve or avoid the in vivo cell adhesion. The bioactive treatment in structure's core is very important to promote the native tissue ingrowth and allow a better recovery. The biopassive treatment is also very important to avoid the formation of adhesion plates between the implant and the surrounding tissues to allow its movement in the physiological space. That movement is essential for fibroblasts proliferation and differentiation during the healing process.

The architecture based on a core/shell system, as shown in FIG. 1, grants to the fibrous structure a specific physical and mechanical behaviour when it is repeatedly mechanically loaded, as happens with a native tendon or ligament in constant usage in the human body. The core is based on several sub-components, namely braided structures parallelly assembled, which are enclosed by a shell.

A simple tubular braid (3), as shown in FIGS. 1 and 2, is a fibrous structure formed by crossing a number of filaments diagonally in such a way that each group of filaments pass alternately over and under a group of filaments laid up in the opposite direction. Due to its structural integrity, durability, design flexibility and precision, braided structures have been used for different critical applications.

Regarding the braiding pattern, which consists of the intersection repeat of the yarn groups, these structures may be classified as diamond (1/1 repeat), regular (2/2 repeat), which is the most used, or Hercules (3/3 repeat), or any derivative. Besides that, the braided structures can even be categorized as biaxial or triaxial, according to the orientation of the constituent filaments. In general, both types of braids have two sets of braider filaments placed in the clockwise and counter clockwise directions (typically each strand aligned in the bias direction), whereas triaxial braids also have an additional set of strands aligned in the direction of braid.

Moreover, the architecture of a braided structure is strongly affected by the number of filaments composing it, by the diameter of those filaments and by braid angle. The braid angle is the angle that each yarn in the braid makes with the braid longitudinal line. The braids architecture influences their porosity level, swelling profile, wicking ability and mostly their mechanical behaviour.

In this invention, the sub-components that compose the core are braided structures (FIG. 2) that may present a diamond, regular or even Hercules braiding pattern. According to the orientation of the constituent filaments, the braids may be biaxial or triaxial. The braid angle may range from 0 until 90°, regardless of the production technique of the structure.

The sub-components that compose the core are braided structures based on polymeric filaments, which may be based on non-degradable polymers such as polypropylene (PP), polyethylene (PE), poly(ethylene terephthalate) (PET) or even polyamide (PA), or by any combination thereof. The diameter of those filaments may range from 5 until 1000 μm.

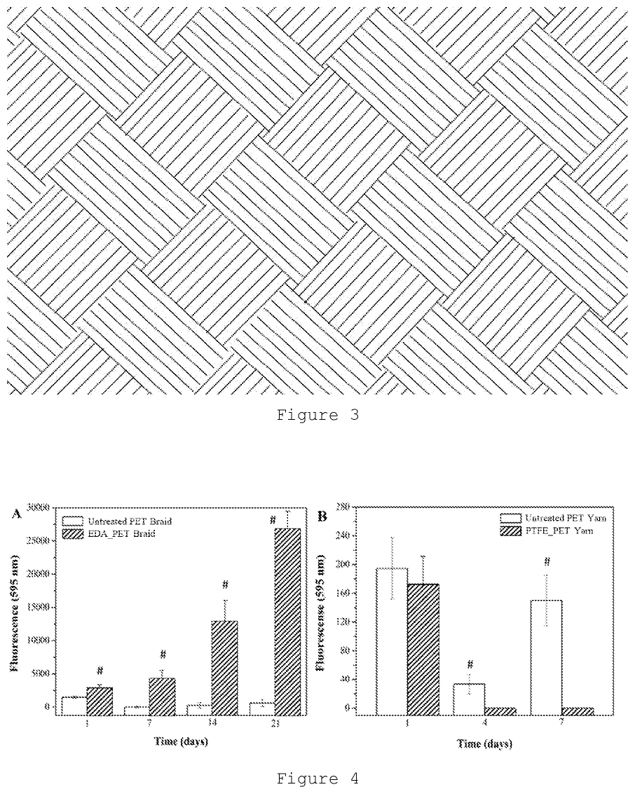

The shell (2) that encloses the core (1) components is based on several braided filaments (4), as shown in FIGS. 1 and 3, which may be based on different non-degradable polymeric filaments, such as polypropylene (PP), polyethylene (PE), poly(ethylene terephthalate) (PET) or even polyamide (PA), or by any combination thereof. The diameter of those filaments may range from 5 until 1000 μm.

Moreover, in order to accomplish a structure partially or totally biodegradable, either the core sub-components or the shell of the present invention may also be composed by different types of biodegradable polymers such as polydioxanone (PDO), poly(glycolic-co-caprolactone) (PGCL), poly(glycolic-co-lactic acid, (PGLA), (poly(lactic acid) (PLA), poly(Lactic-co-glycolic acid) (PLGA), 5 Poly(3-hydroxybutyrate-co-3-hydroxyhexanoate) (PHBHHx), poly(3-hydroxybutyrate) (PHB), Polycaprolactone (PCT), Poly(lactic acid) (PLAs), any reinforced composite based on any of these polymers or by any combination thereof, as polymeric filaments. The diameter of those filaments may range from 5 until 1000 μm.

The structure developed in the present invention presents a non-linear force-strain curve, in elongation-to-failure conditions, appropriate for any tendon or ligament repair, according to data reported on several studies on literature. Once that the load and strain at failure, stiffness and Young's modulus of a tendon or ligament depend on its thickness and collagen content, patient gender, age and physical activity, the fibrous structure of the present invention is able to be properly adapted to repair the function of any injured tendon or ligament.

The level of load at failure of the developed structure is mainly controlled by the number of filaments/braids in core, but the level of strain to failure is mostly influenced by the take-up rate and consequent braid angle of braids that compose the core. So, the structure stiffness level results from a combination of the filaments/braids number in core and the associated braid angle.

For each different tendon or ligament, the number of sub-components composing the structure core or even the number of filaments and/or the braid angle in each sub-component will be adapted in order to obtain a structure with an appropriate mechanical performance, namely regarding the level of stiffness and level of load and strain at failure.

The number of filaments in shell and the braid angle are also adaptable according to the required mechanical parameters.

Besides that, when using a combination of different yarn types, the amount of each type must be also adapted in accordance to the desired mechanical performance depending on the tendon or ligament that is intended to be repaired.

Moreover, the developed architecture presents a viscoelastic behaviour with very promising fatigue and creep resistance according to the demanding requirements for the final application.

The homogeneous and high level of porosity associated to the fibrous architecture of the present invention is also a very promising feature of the developed structure to allow a better cell migration and tissue and blood vessels ingrowth into the fibrous structure, what consequently promotes successful implant integration in vivo.

Moreover, regarding the appropriate interaction of the developed fibrous structure with cells, two distinct and selective surface treatments to be applied on filaments/braids/core/shell composing the structure are also provided by the present invention.

Either to replace a tendon or a ligament, the surface of filaments/braids present in the structure, namely in core, must promote the adhesion and proliferation of cells such as endogenous fibroblasts for new tissue ingrowth. The cells, either endogenous fibroblasts or others that migrate to the structure core come from tendon/ligament tissue ends that remain in physiological space even after injury.

Thus, a bioactive treatment to be applied on those filaments/braids surface of the core is also provided in the present invention.

In one embodiment, the bioactive treatment, aiming to promote cell adhesion, can be based on grafting amine (—NH₂) groups on filaments/braids surface by an aminolysis reaction, in which a molecule is split into two parts by reacting with a molecule of an amine, or by grafting any other compound by any other approach that can promote cell adhesion. Aminolysis has been presented as effective to modify polymeric scaffolds for tissue engineering applications, where the free amino groups were used as a chemical linker to immobilize macromolecules, such as gelatin, chitosan and collagen, or they can directly interact with the extracellular matrix (ECM).

In case of grafting with amine groups, any organic compound with at least two amine groups on its composition can be used as source of amine groups, such as cadaverine, diaminopropane, 1,2-Diaminopropane, 1,3-Diaminopropane, dibutylhexamethylenediamine, N,N′-Dimethyl-1,3-propanediamine, ethylenediamine, diethylenetriamine, hexamethylenediamine, norspermidine, putrescine, spermidine, spermine, triethylenetetramine, tris(2-aminoethyl)amine,

or any combination thereof.

The term “source”, which refers to the organic compound reach in amine groups, in no way excludes the use of two or more such sources or any other compound that can promote cell adhesion.

Some (—NH₂) groups from any source are chemisorbed on polymeric substrates by amide groups formation, while the other(s) amine groups are available to interact with the ECM molecules, improving the interface between the scaffolds surface and surrounding cells. Those amino groups present on the material surface present positive charges which are able to establish electrostatic interactions with the negative charges of cell-surface proteins, promoting the adhesion of cells to the material.

Before the aminolysis reaction, the filaments/braids are exposed to a plasma treatment or any other treatment that creates new functional groups on their surface, such as carboxyl (—COOH) and hydroxyl (—OH), which increase the filaments/braids surface hydrophilicity. The higher hydrophilicity improves the contact between the filaments/braids surface and the source solution. Besides that, the new provided chemical groups are new points of reaction to anchorage more molecules from the source. Thus, the number of amine groups available to interact with cells is also higher.

To endow the structure with increased biocompatibility, a mix of the aforementioned functional groups can be used as well, albeit it is key that free hydroxyl groups on the pre-treated braids/filaments are assured before any bioactive approach.

This approach is only one example of the many bioactive treatments that can be applied to the present technology. Any bioactive treatment implemented should aim towards promoting the adhesion and proliferation of cells in filaments and/or core braids.

In case of tendons/ligaments healing process after injury there is an important limitation, which is the formation of scarring and fibrous paratendinous adhesions. After a tendon/ligament partial or total rupture and consequent surgical procedure, the membrane that surrounds tendon disrupts. This membrane, named as paratenon, is a loose connective tissue layer and its rupture allows granulation tissue and fibroblasts from surrounding tissues to invade the damaged site. Therefore, exogenous cells will predominate over the endogenous tenocytes allowing the surrounding tissues to attach to the damaged tissue resulting in adhesions formation. Those adhesions inhibit the movement of that tissue in its physiological space, what prevents the stress transmission into it, impairing the collagen fibres alignment and consequently a normal tissue function. Moreover, it has been reported that the mechanical loading, inexistent in case of physical immobilization, is essential for tenocytes proliferation and differentiation during the healing process.

Therefore, in case of using the structure of the present invention for the repair of a tendon/ligament, a biopassive treatment to be applied on the filaments and/or braid structure of the shell is also provided in this invention in order to mimic the paratenon membrane.

In one embodiment, the biopassive treatment can be based on a grafting or coating with a hydrophobic and low friction compound or polymer such as polytetrafluoroethylene (PTFE) based coating, which prevents the adhesion of exogenous tenocytes on the implant shell due to the provided surface chemistry, roughness and low surface energy (hydrophobic profile).

Moreover, the non-adhesion of exogenous tenocytes on the structure shell and the low coefficient of friction of the grafting/coating will allow the relative movement of the implant when applied in physiological space, what is essential for tenocytes proliferation, as already mentioned. For a PTFE based coating, any PTFE solution with any concentration composed by nano- or microparticles may be used, water-based or not.

This coating may be applied on shell filaments and/or braids using different techniques, namely by air-atomized spray technique, radio frequency (RF) sputtering, or even by immersion.

This approach is only one example of the many biopassive treatments that can be applied to the present technology. Any biopassive treatment implemented should aim towards preventing the adhesion of cells in filaments and/or shell braids.

Other polymers that can be used for this approach include all the others fluoropolymers-polyvinylfluoride (PVF); polyvinylidene fluoride (PVDF); polychlorotrifluoroethylene (PCTFE); perfluoroalkoxy polymer (PFA); fluorinated ethylene-propylene (FEP); polyethylenetetrafluoroethylene (ETFE); polyethylenechlorotrifluoroethylene (ECTFE); Perfluorinated Elastomer (FFPM); Fluorocarbon (Chlorotrifluoroethylenevinylidene fluoride (FPM); Perfluoropolyether (PFPE); Perfluorosulfonic acid (PFSA); Perfluoropolyoxetane (PFPO).

Moreover, the biopassive approach can be achieved by endowing the structure with superhidrophobicity (contact angle higher than 150°) or superhidrophilicity (contact angle lower than 50).

Regarding the clinical application of the fibrous structure in physiological space, a suture is the best option to anchor the structure to a muscle and/or tendon end. Therefore, knitted/woven assemblies and a system based in a group of needles or any other similar system, where fibres bundles are swaged into muscle, can be used for that purpose. For the anchorage to bone, if a loop or any other similar system is incorporated in the structure end, it may be fixed using polymeric screws.

Moreover, this invention also envisages the hypothesis of performing an in vitro host stem cell seeding on the structure core braids before its implantation on the physiological space. This allows creating in vitro a new tissue layer on the structure filaments surface even before the application of the implant, what can decrease the patient recovery time.

Mesenchymal stem cells (MSCs) are an example of such cells that can be used, which are able to differentiate into fibroblasts. A possible source of those cells is the human adipose tissue, which is ubiquitous and easily obtainable in large quantities under local anesthesia with little patient discomfort. So, it is a potential source from the own patient under treatment with a very low rejection risk. Any other source of those cells from the own patient is also envisaged.

In one embodiment, the core structure must have the necessary number of filaments and braids to allow the core to have a diameter of up to 2 cm.

In one embodiment, the shell structure must have the necessary number of filaments and braids to allow the shell to have a thickness of up to 5 mm.

The core/shell measures are related to the measures of ligaments and tendons of the human body, which also vary within these ranges, so that the presently described core/shell structure can be suitable for their repair.

### Examples

Structural Analysis

Different biaxial braided structures were produced from polypropylene (PP) and polyethylene terephthalate (PET) multifilament yarns with a linear density of 1200 and 1112 dtex respectively, on a vertical braiding machine with 16 carriers, under controlled process conditions. For each yarn type, four different braided structures were produced, using always the same pattern (1/1) but with different yarn numbers (6, 8 and 16) and/or braiding take-up rate (H: 3.94 cm/s and L: 1.44 cm/s), Y stands for yarns (table 1).

TABLE 1

| | | Number of | | | |
|---|---|---|---|---|---|
| | | filaments | | Linear | |
| Polymeric | | (yarn)/yarns | Braids/ | density | Tenacity |
| material | Structure | (braid) | cm | (tex) | (N/tex) |
| PP | Yarn | 137 ± 4 | | 120 | 0.62 |
| | 6YH | 6 | | 679 ± 5 | 0.60 |
| | 8YH | 8 | 1.2 ± 0.1 | 1103 ± 4 | 0.51 |
| | 8YL | 8 | 3.2 ± 0.2 | 1183 ± 5 | 0.48 |
| | 16YH | 16 | 2.2 ± 0.1 | 1862 ± 6 | 0.58 |
| PET | Yarn | 162 ± 4 | | 111 | 0.66 |
| | 6YH | 6 | | 682 ± 3 | 0.56 |
| | 8YH | 8 | 1.4 ± 0.1 | 903 ± 5 | 0.65 |
| | 8YL | 8 | 3.5 ± 0.2 | 943 ± 7 | 0.59 |
| | 16YH | 16 | 2.4 ± 0.1 | 1835 ± 5 | 0.64 |

From optical microscopic images of the braided structures based on PP and PET, it was possible to observe the braids architecture, which are classified as biaxial attending to the orientation of the constituent yarns and as diamond, in case of braids with 6 and 8 yarns, and as regular, when using 16 yarns, attending to the braiding pattern. Moreover, also based on optical microscopic images, the braid angle was calculated, which represents the acute angle that each yarn makes with the braid longitudinal line. During the braiding process the yarns interlace diagonally, meaning that each yarn makes an angle with the structure longitudinal axis, as assigned in FIG. 2, which can be between 1° and 89° but is usually in the range of 30°-80°. This angle is called the braid angle and is the most important geometrical parameter of braided structures. The braid angle of a structure is of course related with the number of braiding points/cm. Therefore, as already discussed, when the yarns number increases or the take-up rate decreases, the number of braids/cm tends to increase leading to a higher braid angle.

Braids Porosity

For both yarns, the porosity level does not present a significant change as the number of yarns or take-up rate changes. Even so, using each one of the yarns, the highest porosity level is observed for the 16YH structure, which is about 88% in case of PP and 85% in case of PET, the porosity level of all produced structures was evaluated and it increased when the yarns number increased to 16, being about 88% in case of PP and 85% in case of PET. The porosity of the textile structures is mainly due to due to the open spaces among the yarns, but also due to smaller spaces among filaments composing each yarn, so when increasing the yarns number, it would be expected to have more open spaces.

After the promoted characterization of all produced braids, it is possible to conclude that the braids architecture actually defines their physical and mechanical behaviour, besides of course the intrinsic physical properties of the yarns that compose them. The number of yarns in the structure and the braiding take-up rate are the main parameters that can be adjusted to construct structures with different architectures, namely with a different braids/cm, diameter, linear density, tenacity, braid angle and porosity level. The wicking ability of braids was dependent on structure pores amount but also on how those pores communicate, which also depends on the architecture, namely how the yarns are arranged and packed in the structure.

Production of Core/Shell Structures

Different textile structures based on a core/shell architecture, using PP or PET yarns, were produced on a vertical braiding machine with 32 carriers. The core is composed by several braids based on PP or PET multifilament yarns, and the shell is also composed by braided PP or PET multifilament yarns (table 2).

TABLE 2

| | | Number of | | | |
|---|---|---|---|---|---|
| | | filaments | | Linear | |
| Polymeric | | (yarn)/yarns | Braid/ | density | Tenacity |
| material | Structure: Braids | (braid) | cm | (tex) | (N/tex) |
| PP | 8YH | 8 | 1.2 ± 0.1 | 1103 ± 4 | 0.51 |
| | 16YH | 16 | 2.2 ± 0.1 | 1862 ± 6 | 0.58 |
| PET | 16YH | 16 | 2.4 ± 0.1 | 1835 ± 5 | 0.64 |

| | | Braids | | Linear | |
|---|---|---|---|---|---|
| | | number/ | Braids/ | density | Tenacity |
| | Structure: core/shell | type in core | cm | (tex) | (N/tex) |
| PP | C16B8YH_S16YL | 16/8YH | 4.2 ± 0.1 | 19566 ± 7 | 0.43 |
| | C16B16YH_S16YL | 16/16YH | 4.0 ± 0.1 | 31716 ± 4 | 0.52 |

The braids that compose the structures core were produced with

a braiding take-up rate of 3.94 cm/s (H) using 8 (8YH) or 16 yarns (16YH), using a vertical braiding machine with 16 carriers. The yarns of the shell were braided using a take-up rate of 1.44 cm/s (L). PP and PET multifilament yarns, with a linear density of 1200 and 1112 dtex respectively.

Core-shell structures with a core composed by 8YH and 16YH braids and a shell of 16YL braids were prepared with PP, which were named as C16B8YH_S16YL and C16B16YH_S16YL respectively. For PET, using a braiding take-up rate of 3.94 cm/s (H) a rope was produced with a core of 22 yarns (22YH) and a shell of 16YL braids, which is named as C22B16YH_S16YL.

The three different core-shell structures presented a non-linear force-strain curve with three different regions as also reported in case of native tendon/ligament tensile curve. The load at failure level of core-shell structures is mainly controlled by the number of yarns/braids in core, but the strain to failure level is mostly influenced by the take-up rate and consequent braid angle of braids that compose the core. Therefore, the core-shell structures stiffness level results from a combination of the yarns/braids number in core and the associated braid angle.

Moreover, the PET_C22B16YH_S16YL core-shell structure revealed a very promising fatigue and creep resistance even for a demanding application as the Achilles tendon according to the demanding requirements even for the final application. The high porosity of the PET structure is also a very important feature of this structure to allow a better cell migration and adhesion, tissue and blood vessels ingrowth into the fibrous structure and promote successful implant integration in vivo.

Treatments

In order to modulate the physicochemical features of a core-shell structures surface two different surface treatments with different purposes (bioactive and biopassive) were studied. One treatment, based on amino groups grafting using for example ethylenediamine (EDA) molecules to be applied in the structure core to allow the improvement of cell adhesion and proliferation, and other treatment, based on a hydrophobic coating such as polytetrafluoroethylene (PTFE) to be applied in the structure shell to avoid the cell adhesion. Both treatments should be optimized in order to reach their purposed goals but without harm the tensile properties.

Regarding the bioactive treatment, the results shown in FIG. 4 refer to PET braids (16YH) samples that before the immersion in EDA (concentration 50% v/v in ethanol; 30 min) were exposed to O₂ plasma activation treatment over 8 min using a power of 100 W, a pressure base of 10 Pa and a pressure work of 80 Pa aiming to create new functional groups on the surface, such as carboxyl (—COOH) and hydroxyl (—OH), which increase the PET surface hydrophilicity to improve the contact with the EDA solution. Moreover, the new (—COOH) groups may be new points of reaction to anchorage more EDA molecules.

Regarding the passive treatment, the results shown in FIG. 4 refer to PET yarns coated with PTFE by air-atomized spray technique using water-based PTFE solution with a concentration of 30 g/L.

The metabolic activity of fibroblasts seeded on the EDA grafted braids (16YH) and PTFE coated yarns was evaluated by the resazurin assay over 21 and 7 days of culture, respectively as shown in FIG. 4. In case of EDA grafted braids the fluorescence level significantly increases over time, being the values much higher than for the untreated braids from day 7 until day 21. For the PTFE coated yarns, the fluorescence value significantly decreases from day 1 to day 4, in which it is about 0, and remains the same at day 7. For all time points, the fluorescence was much lower for the coated yarns than for the untreated PET yarns.

1. A bio-functionalized prosthetic structure comprising a core-shell architecture for partial or total repair of human tendons or ligaments, wherein:
a core of the core-shell architecture comprises braided structures parallelly assembled based on a plurality of biocompatible polymeric filaments, wherein the core comprises a braid angle from 0 to 90° and wherein the core and has a diameter of up to 2 cm;
a shell of the core-shell architecture encloses the core and is a braided structure based on a plurality of biocompatible polymeric filaments, wherein the shell comprises a braid angle from 0 to 90° and wherein the shell has a thickness of up to 5 mm;
the plurality of biocompatible polymeric filaments and/or braids in the core comprise a bioactive surface treatment suitable for cell adhesion and proliferation; and
the plurality of biocompatible polymeric filaments and/or braids in the shell comprise a biopassive surface treatment suitable to avoid the formation of adhesion plates between the prosthetic structure and the surrounding tissues of tendons or ligaments.

2. The bio-functionalized prosthetic structure with core-shell architecture according to claim 1, wherein the plurality of biocompatible polymeric filaments in the core are composed by non-degradable filaments selected from the group consisting of polypropylene (PP), polyethylene (PE), poly (ethylene terephthalate) (PET), polyamide (PA), a reinforced composite based on any of the foregoing polymers, and a combination thereof.

3. The bio-functionalized prosthetic structure with core-shell architecture according to claim 1, wherein the biocompatible polymeric filaments in the core are composed by biodegradable filaments selected from the group consisting of polydioxanone (PDO), poly(glycolic-co-caprolactone) (PGCL), poly(glycolic-co-lactic acid) (PGLA), poly(lactic acid) (PLA), poly(lactic-co-glycolic acid) (PLGA), a reinforced composite based on the foregoing polymers, and a combination thereof.

4. The bio-functionalized prosthetic structure with core-shell architecture according to claim 1, wherein the biocompatible polymeric filaments in the shell are composed by non-degradable filaments selected from the group consisting of polypropylene (PP), polyethylene (PE), poly (ethylene terephthalate) (PET) or polyamide (PA), a reinforced composite based on the foregoing polymers, and a combination thereof.

5. The bio-functionalized prosthetic structure with core-shell architecture according to claim 1, wherein the biocompatible polymeric filaments in the shell are composed by biodegradable filaments selected from the group consisting of polydioxanone (PDO), poly(glycolic-co-caprolactone)(PGCL), poly(glycolic-co-lactic acid) (PGLA), poly(lactic acid)(PLA), poly(lactic-co-glycolic acid) (PLGA), 5Poly(3-hydroxybutyrate-co-3 hydroxyhexanoate) (PHBHHx), poly(3-hydroxybutyrate) (PHB), Polycaprolactone (PCL), a reinforced composite based on the foregoing polymers, and a combination thereof.

6. The bio-functionalized prosthetic structure with core-shell architecture according to claim 1, wherein the diameter of the filaments is within the range of 5-1000 μm.

7. The bio-functionalized prosthetic structure with core-shell architecture according to claim 1, wherein the bioactive surface treatment is based on grafting —NH₂ groups on the filaments or braids surface of the biocompatible polymeric.

8. The bio-functionalized prosthetic structure with core-shell architecture according to claim 1, wherein the bioactive surface treatment is based on a functional group grafting after a surface treatment that grants —OH or deprotonated —OH groups to the polymeric structure.

9. The bio-functionalized prosthetic structure with core-shell architecture according to claim 1, wherein the biopassive surface treatment is based on a polytetrafluoroethylene-based coating, or any perfluoro-polymer coating.

10. The bio-functionalized prosthetic structure with core-shell architecture according to claim 1, wherein the biopassive surface treatment is based on a superhydrophobic having a contact angle ≥150° or a superhydrophilic having a contact angle ≤5° compounds.

11. The bio-functionalized prosthetic structure with core-shell architecture according to claim 1, wherein the braiding patterns are diamond 1/1 repeat, regular 2/2 repeat or Hercules 3/3 repeat or any derivative.

12. The bio-functionalized prosthetic structure with core-shell architecture according to claim 1, wherein the braids are biaxial or triaxial.
